(19) United States
(12) Patent Application Publication
(10) Pub. No.: US 2015/0360026 A1
(43) Pub. Date: Dec. 17, 2015

(54) SYSTEMS AND METHODS FOR SYNCHRONIZING THE STIMULATION OF CELLULAR FUNCTION IN TISSUE

(71) Applicant: Timothy Andrew Wagner, Somerville, MA (US)

(72) Inventor: Timothy Andrew Wagner, Somerville, MA (US)

(21) Appl. No.: 14/241,168

(22) PCT Filed: Aug. 3, 2012
(86) PCT No.: PCT/US12/49466

§ 371 Date: Apr. 17, 2014

Related U.S. Application Data
(60) Provisional application No. 61/531,338, filed on Sep. 6, 2011.

(51) Int. Cl. A61N 1/36; A61N 7/00

(52) U.S. Cl. CPC A61N 1/36014; A61N 7/00; A61N 2007/0021

(57) ABSTRACT

The present invention generally relates to systems and methods for stimulating tissue using synchronized energy from multiple sources. In certain embodiments, the invention provides a system for stimulating tissue that includes a first energy source, a second energy source, and a synchronizing element that synchronizes the two energies such that the combined effect stimulates the tissue.

### RELATED APPLICATION

The present application claims the benefit of and priority to U.S. provisional patent application Ser. No. 61/531,338, filed Sep. 6, 2011, the content of which is incorporated by reference herein in its entirety.

### FIELD OF THE INVENTION

The invention generally relates to systems and methods for stimulating cellular function in biological tissue.

### BACKGROUND

Stimulation of tissue in humans and other animals is used in a number of clinical applications as well as in clinical and general biological research. In particular, stimulation of neural tissue has been used in the treatment of various diseases including Parkinson's disease, depression, and intractable pain. The stimulation may be applied invasively, e.g., by performing surgery to remove a portion of the skull and implanting electrodes in a specific location within brain tissue, or non-invasively, e.g., transcranial direct current stimulation and transcranial magnetic stimulation. Stimulation can also be enacted via combined energy methods, such as through the combined application of mechanical energy (such as generated via an ultrasound transducer) and electrical energy (such as generated via an electrode placed on the scalp), where the combined electromechanical energy can be used to stimulate the brain invasively or noninvasively.

A problem with tissue stimulation via combined energies is an inability to effectively synchronize the individual energies for effective tissue stimulation. The lack of synchronization makes it impractical to effectively combine energies for stimulation of tissues for certain effects, where the appropriate control over the relative energy characteristics is necessary to stimulate desired cellular function and/or tissue response.

### SUMMARY

The present invention generally relates to systems and methods for stimulating tissue using combined energy types. Systems and methods of the invention use a synchronizing element to synchronize multiple energies that are applied to the tissue. In this manner, the stimulation can be effectively applied to desired cells and/or tissue, which aids in dosing of the stimulation, in characterizing the safety parameters of stimulation, and in maximizing the therapeutic effect of stimulation.

In certain aspects, the invention provides systems for stimulating tissue that include a first energy source, a second energy source, and a synchronization element that synchronizes the first and second energy source so that the first and second energy sources are coordinated relative to each other in energy magnitude, energy position, energy dynamic behavior (i.e., behavior as a function of time), energy static behavior, energy behavior in the frequency domain, energy phase, orientation/direction of energy fields (i.e., vector behavior), duration of energy application (in single or multiple sessions), and/or type/composition of energy. In certain embodiments, the synchronization is tuned for a specific cell and/or tissue type for a particular response. The synchronization of energies can be applied to affect or be synchronized with functions of cell(s), tissue(s), network(s), organs(s), and organism(s).

Methods of the invention can be implemented during stimulation, after stimulation, or before stimulation (such as where synchronization planning could take place via simulation).

Any type of energies known in the art may be used with methods of the invention. In certain embodiments, the first type of energy is a mechanical energy field, such as that produced by an ultrasound device. The mechanical field may be pulsed, time varying, or pulsed a plurality of time with each pulse being for a different length of time. In certain embodiments, the ultrasound device includes a focusing element so that the mechanical field may be focused. In certain embodiments, the second type of energy is an electrical energy field, such as that produced by placing at least one electrode in or near the tissue. The electric field may be pulsed, time varying, pulsed a plurality of time with each pulse being for a different length of time, or time invariant. In certain embodiments, the electrical energy is focused, and focusing may be accomplished based upon placement of electrodes. In still other embodiments, both the electrical and mechanical fields are focused. In still other embodiments, mechanical, chemical, optical, electromagnetic, and/or thermal energy may be combined.

The multiple energy sources may be applied to any tissue. In certain embodiments, the first and second energy sources are applied to a structure or multiple structures within the brain or the nervous system such as the dorsal lateral prefrontal cortex, any component of the basal ganglia, nucleus accumbens, gastric nuclei, brainstem, thalamus, inferior colliculus, superior colliculus, periaqueductal gray, primary motor cortex, supplementary motor cortex, occipital lobe, Brodmann areas 1-48, primary sensory cortex, primary visual cortex, primary auditory cortex, amygdala, hippocampus, cochlea, cranial nerves, cerebellum, frontal lobe, occipital lobe, temporal lobe, parietal lobe, sub-cortical structures, and spinal cord. In particular embodiments, the tissue is neural tissue, and the affect of the stimulation alters neural function past the duration of stimulation.

Another aspect of the invention provides a method for stimulating tissue that involves providing a first type of energy to a region of tissue, providing a second type of energy to the region of tissue, and synchronizing the energies so that the first and second types of energy are effectively combined in the region of tissue, such that the combined effect stimulates the tissue.

### BRIEF DESCRIPTION OF THE DRAWINGS

The above-mentioned and other features and objects of this invention, and the manner of attaining them, will become more apparent and the invention itself will be better understood by reference to the following description of embodiments of the invention taken in conjunction with the accompanying drawings, wherein:

FIG. 1 is a plan view of one embodiment of an apparatus for stimulating biological tissue constructed in accordance with the principles of the present disclosure;

FIG. 2 is a top plan view of an exemplary embodiment of an apparatus for stimulating biological tissue implementing a chemical source for altering permittivity constructed in accordance with the principles of the present disclosure;

FIG. 3 is a top plan view of an exemplary embodiment of an apparatus for stimulating biological tissue implementing a radiation source for altering permittivity constructed in accordance with the principles of the present disclosure; and

FIG. 4 is a top plan view of another exemplary embodiment of an apparatus for stimulating biological tissue implementing an optical beam for altering permittivity constructed in accordance with the principles of the present disclosure.

### DETAILED DESCRIPTION

It is envisioned that the present disclosure may be used to stimulate biological tissue in-vivo comprising the application of at least two energy types where the synchronization of the energies is necessary for effective stimulation of the combined energies.

The exemplary embodiments of the apparatuses and methods disclosed can be employed in the area of neural stimulation, where synchronized combined energies can be used for directly stimulating neurons, depolarizing neurons, hyperpolarizing neurons, modifying neural membrane potentials, altering the level of neural cell excitability, and/or altering the likelihood of a neural cell firing (during and after the period of stimulation). Likewise, methods for stimulating biological tissue may also be employed in the area of muscular stimulation, including cardiac stimulation, where synchronized combined energies can be used to alter muscular activity via direct stimulation, depolarizing muscle cells, hyperpolarizing muscle cells, modifying membrane potentials, altering the level of muscle cell excitability, and/or altering the likelihood of cell firing (during and after the period of stimulation). Likewise, methods for stimulating tissue can be used in the area of cellular metabolism, physical therapy, drug delivery, and gene therapy. Furthermore, stimulation methods described herein can result in or influence tissue growth (such as promoting bone growth or interfering with a tumor).

The present invention generally relates to systems and methods for stimulating tissue using combined energy types. Systems and methods of the invention use a synchronizing element to synchronize multiple energies that are applied to the tissue. In this manner, the stimulation can be effectively applied to desired cells/tissue, which aids in dosing of the stimulation, in characterizing the safety parameters of stimulation, and in maximizing the therapeutic effect of stimulation.

In certain aspects, the invention provides systems for stimulating tissue that include a first energy source, a second energy source, and a synchronization element that synchronizes the first and second energy source so that the first and second energy sources are coordinated relative to each other in energy magnitude, energy position, energy dynamic behavior (i.e., behavior as a function of time), energy static behavior, energy behavior in the frequency domain, energy phase, orientation/direction of energy fields (i.e., vector behavior), duration of energy application (in single or multiple sessions), and/or type/composition of energy. In certain embodiments, the synchronization is tuned for a specific cell and/or tissue type for a particular response. Methods of the invention can be implemented during stimulation, after stimulation, or before stimulation (such as where synchronization planning could take place via simulation). Any type of energies known in the art may be used with methods of the invention, such as mechanical, chemical, optical, electromagnetic, and/or thermal energy may be combined. The energies may be provided by invasive and/or noninvasive sources (and/or with multiple energy sources and/or multi-energy source elements (e.g., a transducer that provides both energies simulataneously)).

The embodiments outlined herein for synchronizing energies for stimulation can be integrated (either through feedback control methods or passive monitoring methods) with imaging modalities, physiological monitoring methods/devices, diagnostic methods/devices, and biofeedback methods/devices (such as those described in co-owned and co-pending U.S. patent application Ser. No. 13/162,047, the content of which is incorporated by reference herein in its entirety). The embodiments outlined herein for synchronizing energies for stimulation can be integrated with or used to control the stimulation source properties (such as number, material properties, position (e.g., location and/or orientation relative to tissue to be stimulated and/or other sources or components to be used in the stimulation procedure) and/or geometry (e.g., size and/or shape relative to tissue to be stimulated and/or other sources or components to be used in the stimulation procedure)), the stimulation energy waveform (such as temporal behavior, intensity, and/or duration of application), properties of interface components (such as those outlined in (U.S. patent application number 2010/0070006) and for example position, geometry, and/or material properties of the interface materials), and/or properties of focusing or targeting elements (such as those outlined in (co-owned and co-pending U.S. patent application Ser. No. 13/169,288, the content of which is incorporated by reference herein in its entirety) and for example position, geometry, and/or material properties of the interface materials) used during stimulation. Furthermore, the synchronization methods outlined herein, can also be integrated with methods which assess, control, and optimize stimulation based on tissue filtering properties (such as those outlined in (co-owned and co-pending U.S. patent application Ser. No. 13/216,282 the content of which is incorporated by reference herein in its entirety)).

In certain embodiments, methods of the invention can be accomplished with computers, mobile devices, dedicated chips or circuitry (e.g., in control system of stimulator or integrated imaging device or external dose controller), remote computational systems accessed via network interfaces, and/or computational devices known in the art. Methods of the invention can be accomplished with software for performing various computer-implemented processing operations such as any or all of the various operations, functions, and capabilities described herein. In certain embodiments, the processing operations include accessing a database of source, tissue, organ, network, organism, and/or cellular properties that can be stored in any form of computer storage.

The term “computer-readable medium” is used herein to include any medium capable of storing data and/or storing or encoding a sequence of computer-executable instructions or code for performing the processing operations described herein. The media and code can be those specially designed and constructed for the purposes of the invention, or can be of the kind well known and available to those having ordinary skill in the computer and/or software arts. Examples of computer-readable media include computer-readable storage media such as: magnetic media such as fixed disks, floppy disks, and magnetic tape; optical media such as Compact Disc-Read Only Memories (“CD-ROMs”) and holographic devices; magneto-optical media such as floptical disks; memory sticks “flash drives” and hardware devices that are specially configured to store and execute program code, such as Application-Specific Integrated Circuits (“ASICs”), Programmable Logic Devices (“PLDs”), Read Only Memory (“ROM”) devices, and Random Access Memory (“RAM”) devices. Examples of computer-executable program instructions or code include machine code, such as produced by a compiler, and files containing higher level code that are executed by a computer using an interpreter. For example, an embodiment of the invention may be implemented using Java, C++, or other programming language and development tools. Additional examples of instructions or code include encrypted code and compressed code. Other embodiments of the invention can be implemented in whole or in part with hardwired circuitry in place of, or in combination with, program instructions/code.

The software can run on a local computer or a remote computer accessed via network connections. The computer may be a desktop computer, a laptop computer, a tablet PC, a cellular telephone, a Blackberry, or any other type of computing device. The computer machine can include a CPU, a ROM, a RAM, an HDD (hard disk drive), an HD (hard disk), an FDD (flexible disk drive), an FD (flexible disk), which is an example of a removable recording medium, a display, an I/F (interface), a keyboard, a mouse, a scanner, and a printer. These components are respectively connected via a bus and are used to execute computer programs described herein. Here, the CPU controls the entire computer machine. The ROM stores a program such as a boot program. The RAM is used as a work area for the CPU. The HDD controls the reading/writing of data from/to the HD under the control of the CPU. The HD stores the data written under the control of the HDD. The FDD controls the reading/writing of data from/to the FD under the control of the FDD. The FD stores the data written under the control of the FDD or causes the computer machine to read the data stored in the FD. The removable recording medium may be a CD-ROM (CD-R or CD-RW), a DVD (Digital Versatile Disk), a memory card or the like instead of the FD. The display displays data such as a document, an image and functional information, including a cursor, an icon and/or a toolbox, for example. The display may be a CRT, a TFT liquid crystal display, or a plasma display, for example. The I/F may be connected to the network such as the Internet via a communication line and is connected to other machines over the network. The I/F takes charge of an internal interface with the network and controls the input/output of data from/to an external machine. A modem or a LAN adapter, for example, may be adopted as the I/F. The keyboard includes keys for inputting letters, numbers and commands and is used to input data. The keyboard may be a touch-panel input pad or a numerical keypad. The mouse is used to move a cursor to select a range to move or change the size of a window. A trackball or joystick, for example, may be used as a pointing device if it has the same functions.

Components used with methods of the invention are fabricated from materials suitable for a variety medical applications, such as, for example, polymerics, gels, films, and/or metals, depending on the particular application and/or preference. Semi-rigid and rigid polymerics are contemplated for fabrication, as well as resilient materials, such as molded medical grade polyurethane, as well as flexible or malleable materials. The motors, gearing, electronics, power components, electrodes, and transducers of the method may be fabricated from those suitable for a variety of medical applications. The method according to the present disclosure may also include circuit boards, circuitry, processor components, etc. for computerized control. One skilled in the art, however, will realize that other materials and fabrication methods suitable for assembly and manufacture, in accordance with the present disclosure, also would be appropriate.

The following discussion includes a description of the components and exemplary methods for synchronizing the energy fields in biological tissues to affect the resulting tissue response in accordance with the principles of the present disclosure. Alternative embodiments are also disclosed. Methods are disclosed for synchronizing the energy fields, such as electromagnetic (e.g., electrical, magnetic energies), chemical, mechanical, thermal, optical, and/or combined energy fields (e.g. electromechanical (i.e., with electrical energy and mechanical energy)). Reference will now be made in detail to the exemplary embodiments of the present disclosure illustrated in the accompanying figures.

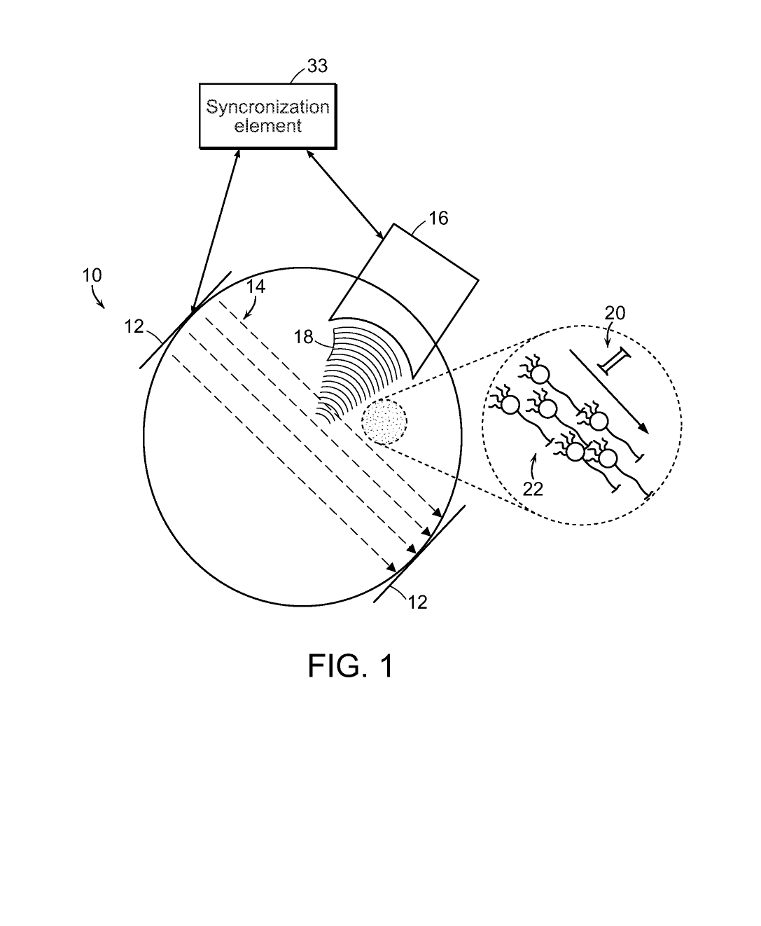

Turning now to FIG. 1, which illustrates an exemplary embodiment of an apparatus 10 to alter currents, e.g., amplify, focus, alter direction, and/or attenuate in the presence of an applied electric field or applied current source by the combined application of a mechanical field within a biological material to stimulate the biological cells and/or tissue in accordance with the present disclosure. For example, the apparatus 10 illustrated in FIG. 1 according to the present disclosure may be applied to the area of neural stimulation. An initial source electric field 14 results in a current in the tissue. The electric field 14 is created by an electric source, current or voltage source. As described in further detail below, the permittivity of the tissue is altered relative to the electric field, for example by a mechanical field, thereby generating an additional displacement current.

Electrodes 12 are applied to the scalp and generate a low magnitude electric field 14 over a large brain region. While electrodes 12 are used and applied to the scalp in this exemplary embodiment, it is envisioned that the electrodes may be applied to a number of different areas on the body including areas around the scalp. It is also envisioned that one electrode may be placed proximal to the tissue being stimulated and the other distant, such as one electrode on the scalp and one on the thorax. It is further envisioned that electric source could be mono-polar with just a single electrode, or multi-polar with multiple electrodes. Similarly, the electric source may be applied to tissue via any medically acceptable medium. It is also envisioned that means could be used where the electric source does not need to be in direct contact with the tissue, such as for example, inductive magnetic sources where the entire tissue region is placed within a large solenoid generating magnetic fields or near a coil generating magnetic fields, where the magnetic fields induce electric currents in the tissue.

The electric source may be direct current (DC) or alternating current (AC) and may be applied inside or outside the tissue of interest. Additionally, the source may be time varying. Similarly, the source may be pulsed and may be comprised of time varying pulse forms. The source may be an impulse. Also, the source according to the present disclosure may be intermittent. The electric field source could also work as a component in the imaging process. The electrical field may be of any arbitrary shape as synchronized with the mechanical field 18.

A mechanical source such as an ultrasound source 16 is applied on the scalp and provides concentrated acoustic energy 18, i.e., mechanical field to a focused region of neural tissue, affecting a smaller number of neurons 22 than affected by the electric field 14, by the mechanical field 18 altering the tissue permittivity relative to the applied electric field 14, and thereby generating the altered current 20. The mechanical source may be any acoustic source such as an ultrasound device. Generally, such device may be a device composed of electromechanical transducers capable of converting an electrical signal to mechanical energy such as those containing piezoelectric materials, a device composed of electromechanical transducers capable of converting an electrical signal to mechanical energy such as those in an acoustic speaker that implement electromagnets, a device in which the mechanical source is coupled to a separate mechanical apparatus that drives the system, or any similar device capable of converting chemical, plasma, electrical, nuclear, or thermal energy to mechanical energy and generating a mechanical field.

Furthermore, the mechanical field could be generated via an ultrasound transducer that could be used for imaging tissue. The mechanical field may be coupled to tissue via a bridging medium, such as a container of saline to assist in the focusing or through gels and/or pastes which alter the acoustic impedance between the mechanical source and the tissue. The mechanical field may be time varying, pulsed, an impulse, or may be comprised of time varying pulse forms. It is envisioned that the mechanical source may be applied inside or outside of the tissue of interest. There are no limitations as to the frequencies that can be applied via the mechanical source, however, exemplary mechanical field frequencies range from the sub kHZ to 1000s of MHz. The mechanical field may be of any arbitrary shape as synchronized with the electrical field. Additionally, multiple transducers providing multiple mechanical fields with similar or differing frequencies, and/or similar or different mechanical field waveforms may be used—such as in an array of sources like those used in focused ultrasound arrays. Similarly, multiple varied electric fields could also be applied. The combined fields, electric and mechanical, may be controlled intermittently to cause specific patterns of spiking activity or alterations in neural excitability. For example, the device may produce a periodic signal at a fixed frequency, or high frequency signals at a pulsed frequency to cause stimulation at pulse frequencies shown to be effective in treating numerous pathologies. Such stimulation waveforms may be those implemented in rapid or theta burst TMS treatments, deep brain stimulation treatments, epidural brain stimulation treatments, spinal cord stimulation treatments, or for peripheral electrical stimulation nerve treatments. The ultrasound source may be placed at any location relative to the electrode locations, i.e., within, on top of, below, or outside the same location as the electrodes as long as components of the electric field and mechanical field are in the same region. The locations of the sources should be relative to each other such that the fields intersect relative to the tissue and cells to be stimulated, or to direct the current alteration relative to the cellular components being stimulated.

The apparatus and method according to the present disclosure generates capacitive currents via permittivity alterations, which can be significant in magnitude, especially in the presence of low frequency applied electric fields. Tissue permittivities in biological tissues are much higher than most other non biological materials, especially for low frequency applied electric fields where the penetration depths of electric fields are highest. This is because the permittivity is inversely related to the frequency of the applied electric field, such that the tissue permittivity magnitude is higher with lower frequencies. For example, for electric field frequencies below 100,000 Hz, brain tissue has permittivity magnitudes as high as or greater than 10̂8 (100,000,000) times the permittivity of free space (8.854*10̂-12 farad per meter), and as such, minimal local perturbations of the relative magnitude can lead to significant displacement current generation. As the frequency of the electric field increases, the relative permittivity decreases by orders of magnitude, dropping to magnitudes of approximately 10̂3 times the permittivity of free space (8.854*10̂-12 farad per meter) for electric field frequencies of approximately 100,000 Hz. Additionally, by not being constrained to higher electric field frequencies, the method according to the present disclosure is an advantageous method for stimulating biological tissue due to lowered penetration depth limitations and thus lowered field strength requirements. Additionally, because displacement currents are generated in the area of the permittivity change, focusing can be accomplished via the ultrasound alone. For example, to generate capacitive currents via a permittivity perturbation relative to an applied electric field as described above, broad DC or a low frequency electric source field well below the cellular stimulation threshold is applied to a brain region but stimulation effects are locally focused in a smaller region by altering the tissue permittivity in the focused region of a mechanical field generated by a mechanical source such as an ultrasound source. This could be done noninvasively with the electrodes and the ultrasound device both placed on the scalp surface such that the fields penetrate the tissue surrounding the brain region and intersect in the targeted brain location, or with one or both of the electrodes and/or the ultrasound device implanted below the scalp surface (in the brain or any of the surrounding tissue) such that the fields intersect in the targeted region.

A displacement current is generated by the modification of the permittivity in the presence of the sub threshold electric field and provides a stimulatory signal. In addition to the main permittivity change that occurs in the tissues, which is responsible for stimulation (i.e., the generation of the altered currents for stimulation), a conductivity change could also occur in the tissue, which secondarily alters the ohmic component of the currents. In a further embodiment, the displacement current generation and altered ohmic current components may combine for stimulation. Generally, tissue conductivities vary slightly as a function of the applied electric field frequency over the DC to 100,000 Hz frequency range, but not to the same degree as the permittivities, and increase with the increasing frequency of the applied electric field. Additionally in biological tissues, unlike other materials, the conductivity and permittivity do not show a simple one-to-one relationship as a function of the applied electric field frequency. The permittivity ranges are as discussed above.

Although the process described may be accomplished at any frequency of the applied electric field, the method in an exemplary embodiment is applied with lower frequency applied electric fields due to the fact the permittivity magnitudes of tissues, as high as or greater than 10̂8 times the permittivity of free space, and the electric field penetration depths are highest for low frequency applied electric fields. Higher frequency applied electric fields may be less desirable as they will require greater radiation power to penetrate the tissue and/or a more pronounced mechanical source for permittivity alteration to achieve the same relative tissue permittivity change, i.e., at higher applied electric field frequencies the permittivity of the tissue is lower and as such would need a greater overall perturbation to have the same overall change in permittivity of a tissue as at a lower frequency. Applied electric field frequencies in the range of DC to approximately 100,000 Hz frequencies are advantageous due to the high tissue permittivity in this frequency band and the high penetration depth for biological tissues at these frequencies. In this band, tissues are within the so called ‘alpha dispersion band’ where relative tissue permittivity magnitudes are maximally elevated (i.e., as high as or greater than 10̂8 times the permittivity of free space). Frequencies above approximately 100,000 to 1,000,000 Hz for the applied electric fields are still applicable for the method described in generating displacement currents for the stimulation of biologic cells and tissue, however, both the tissue permittivity and penetration depth are limited for biological tissues in this band compared to the previous band but displacement currents of sufficient magnitude can still be generated for some applications. In this range, the magnitude of the applied electric field will likely need to be increased, or the method used to alter the permittivity relative to the applied electric field increased to bring about a greater permittivity change, relative to the tissue's permittivity magnitude for the applied electric field frequency. Additionally, due to potential safety concerns for some applications, it may be necessary to limit the time of application of the fields or to pulse the fields, as opposed to the continuous application that is possible in the prior band. For tissues or applications where the safety concerns preclude the technique in deeper tissues, the technique could still be applied in more superficial applications in a noninvasive manner or via an invasive method. Higher frequency applied electric fields, above 1,000,000 to 100,000,000 Hz, could be used in generating displacement currents for the stimulation of biologic cells and tissue. However, this would require a more sufficient permittivity alteration or electromagnetic radiation, and as such is less than ideal in terms of safety than the earlier bands. For frequencies of the applied electric field above 100,000,000 Hz, biologic cell and tissue stimulation may still be possible, but may be limited for specialized applications that require less significant displacement currents.

The focus of the electric and mechanical fields to generate an altered current according to the present disclosure may be directed to various structures within the brain or nervous system including but not limited to dorsal lateral prefrontal cortex, any component of the basal ganglia, nucleus accumbens, gastric nuclei, brainstem, thalamus, inferior colliculus, superior colliculus, periaqueductal gray, primary motor cortex, supplementary motor cortex, occipital lobe, Brodmann areas 1-48, primary sensory cortex, primary visual cortex, primary auditory cortex, amygdala, hippocampus, cochlea, cranial nerves, cerebellum, frontal lobe, occipital lobe, temporal lobe, parietal lobe, sub-cortical structures, spinal cord, nerve roots, sensory organs, and peripheral nerves.

The focused tissue may be selected such that a wide variety of pathologies may be treated. Such pathologies that may be treated include but are not limited to Multiple Sclerosis, Amyotrophic Lateral Sclerosis, Alzheimer's Disease, Dystonia, Tics, Spinal Cord Injury, Traumatic Brain Injury, Drug Craving, Food Craving, Alcohol Craving, Nicotine Craving, Stuttering, Tinnitus, Spasticity, Parkinson's Disease, Parkinsonianism, Obsessions, Depression, Schizophrenia, Bipolar Disorder, Acute Mania, Catonia, Post-Traumatic Stress Disorder, Autism, Chronic Pain Syndrome, Phantom Limb Pain, Epilepsy, Stroke, Auditory Hallucinations, Movement Disorders, Neurodegenerative Disorders, Pain Disorders, Metabolic Disorders, Addictive Disorders, Psychiatric Disorders, Traumatic Nerve Injury, and Sensory Disorders. Furthermore, electric and mechanical fields to generate an altered current may be focused on specific brain or neural structures to enact procedures including sensory augmentation, sensory alteration, anesthesia induction and maintenance, brain mapping, epileptic mapping, neural atrophy reduction, neuroprosthetic interaction or control with nervous system, stroke and traumatic injury neurorehabilitation, bladder control, assisting breathing, cardiac pacing, muscle stimulation, and treatment of pain syndromes, such as those caused by migraine, neuropathies, and low-back pain; or internal visceral diseases, such as chronic pancreatitis or cancer. The methods herein could be expanded to any form of arthritis, impingement disorders, overuse injuries, entrapment disorders, and/or any muscle, skeletal, or connective tissue disorder which leads to chronic pain, central sensitization of the pain signals, and/or an inflammatory response.

In the focused region of tissue to which the mechanical fields are delivered, the excitability of individual neurons can be heightened to the point that the neurons can be stimulated by the combined fields, or be affected such as to cause or amplify the alteration of the neural excitability caused by the altered currents, either through an increase or decrease in the excitability of the neurons. This alteration of neural excitability can last past the duration of stimulation and thus be used as a basis to provide lasting treatment. Additionally, the combined fields can be provided in multiple, but separate sessions to have a summed, or carry-over effect, on the excitability of the cells and tissue. The combined fields can be provided prior to another form of stimulation, to prime the tissue making it more or less susceptible to alternate, follow-up forms of stimulation. Furthermore, the combined fields can be provided after an alternate form of stimulation, where the alternate form of stimulation is used to prime the tissue to make it more or less susceptible to the form of stimulation disclosed herein. Furthermore, the combined fields could be applied for a chronic period of time. The energies can also be applied where the primary focus is not in the generation of a displacement current, such as where the fields can be used for different aspects of stimulation, such as the mechanical field accomplishing one aspect of stimulation while the electrical field accomplishes a separate aspect of stimulation, yet the combined effects of stimulation have a desired outcome not possible by either alone.

A synchronization element, 33 in FIG. 1, is connected to the electrodes 12 (and/or to the source driving the field generated by the electrodes) and the mechanical source 16 (and/or to the source driving the field generated by the mechanical field) to synchronize the application of the electrical field 14 and the mechanical field 18. The synchronization element 33 is implemented to synchronize the combined energies relative to each other in energy magnitude, energy position, energy dynamic behavior (i.e., behavior as a function of time), energy static behavior, energy behavior in the frequency domain, energy phase, orientation/direction of energy fields (i.e., vector behavior), duration of energy application (in single or multiple sessions), and/or type/composition of energy (such as for the electromagnetic energy, the energy stored in the electric field, the magnetic field, or the dissipative current component (such as could be described with a Poynting Vector)). The synchronization element is used to effectively stimulate the targeted cells (and/or tissue), based on controlling the energy application relative to the cells' (and/or tissues') desired response profile.

For example, the synchronization element, 33, can be used to control the timing of electromagnetic energy pulses relative to the mechanical energy pulses, such that they can effectively stimulate the neural targets, such as for example coordinating the initiation and/or conclusion of the energy pulses. The synchronized timing can be based on any desired timing criteria, such as for example a targeted tissue(s), cell's, or cells' (and/or sub components′): electrical, mechanical, and/or electromechanical response(s); the anticipated energy field dynamics in the tissue (such as for example controlling the timing such that the amplitude of the two fields is maximum at the same time); electrophysiological criteria that influences that stimulation response of the targeted tissues (such as for example coordinating the timing of the energy application such that is synchronized with blood flow in the targeted tissue); synchronizing the timing of the energies based on feedback, imaging, or information from diagnostic procedures (such as those described in co-owned and co-pending U.S. patent application Ser. No. 13/162,047, the content of which is incorporated by reference herein in its entirety); and/or synchronizing the timing relative to aspects of tissue filtering (and/or the cellular and tissue response) such as those described in (such as those described in co-owned and co-pending U.S. patent application Ser. No. 13/216,282, the content of which is incorporated by reference herein in its entirety).

For example, an square pulse of electromagnetic energy can be applied in synch with an sinusoidal pulse of mechanical energy, such that their timing is controlled relative to each other, such that the energy field pulses are maximum at the same time in the region of a targeted cell, and that the energy pulses are applied for just the appropriate time such that the cell responds as desired (such as in enacting stimulation to initiate an action potential from a neural cell, but where the stimulation pulses are not being active long enough in duration such that the cell responds with multiple action potentials, such as for example providing the stimulation for a duration shorter than the absolute refractory period of the cell).

As another example, the synchronization element, 33, can be implemented such that the electrical field and mechanical energy are synchronized in the frequency domain, such as for example coordinating the application of electromagnetic energy at a frequency, calibrated based on the electrical impedance of a tissue as a function of frequency, and a mechanical energy at a particular frequency based on the mechanical impedance of a tissue and further synchronized to elicit the desired neural response (for example one could provide transcranial pulses of electromagnetic energy, with the power centered at 50 Hz based on the high permittivity of brain tissue and high penetration depth of the energy in the brain tissue and surrounding tissues (such as skull and muscle), synchronized with transcranial pulses of mechanical energy centered at 750 kHz based on the mechanical impedance of the skull and the ease of transmission through the skull at this frequency, yet provided for brief periods of time and pulsed on and off at 20 Hz to excite neural targets in the motor cortex).

As another example, the synchronization element, 33, can be implemented to synchronize the mechanical energy relative to the electrical energy in phase (for example if electrical and mechanical sinusoidal energy waveforms were applied to the targeted tissue, the synchronizing element can be used to keep the waveforms 90 degrees out of phase (or any arbitrary angle dependent on the desired stimulation effect on the cells/tissue)). As another example, the synchronization element can be used to synchronize the energy fields in direction, time, and position; such as for example the synchronization element, 33, can control the direction of the electrical and mechanical fields relative to each other such that they are orientated together along a particular axis of the targeted cell (such as along the axis of axon when stimulating a neural cell) at the same time.

In certain embodiments, the synchronization element, 33, is tuned for a specific cell and/or tissue type for a particular response. For example, one can synchronize the fields such that they can stimulate neural cells to generate a single action potential (for example by stepping on the electrical and mechanical fields on at the same time with a certain waveform to cause the cell to spike (i.e., generate an action potential), and keeping the synchronized energy active for a period shorter than period where the cell can fire a second spike (e.g., a period shorter than the absolute refractory period of the cell)).

As another example, if one was stimulating cardiac tissue with electromechanical fields, one can synchronize the electromechanical fields relative to each other and relative to cardiac pacing cell activity. As another example, one can enact theta burst type stimulation patterns such as for example by providing synchronized electrical and mechanical pulses: in the intermittent theta burst stimulation pattern (iTBS), a 2 s train of TBS is repeated every 10 s for a total of 190 s; or in the intermediate theta burst stimulation paradigm (imTBS), a 5 s train of TBS is repeated every 15 s for a total of 110 s; or in the continuous theta burst stimulation paradigm (cTBS), a 40 s train of uninterrupted TBS is given (600 pulses).

Similarly the synchronization element can be used to enact tetanic stimulation such as with a rapid (e.g., 30-, 50-, 100-, 200-Hz) synchronized delivery of electromechanical pulses such as to assess neural blocks used during anesthesia. As another example, synchronized energy pulses at 1 Hz for a period of time can be used to inhibit neural activity during and after stimulation, such as in the motor cortex, yet pulse rates above 10 Hz can be used to facilitate neural activity during and after stimulation, such as in the motor cortex (such for example using synchronized waveforms to mimic those found during Transcranial Magnetic Stimulation. Any known stimulation pattern in the art can be applied by controlling the combined energies with the synchronization element.

The synchronization element, 33, can be used to synchronize the combined energies to a cell(s), tissue(s), network(s), organ(s), and/or organism(s). For example, the synchronization of energies can be applied to affect or be synchronized with cellular processes and/or functions, such as a cells′: membrane potentials, ionic distributions in and around the cell, firing rates, level of cellular excitability, channel dynamics, membrane dynamics, genetic processes, configurations of channels, timing or intensity of cellular processes, synaptic plasticity, cell position, cell exocytosis, membrane impedances, channel impedances, cellular transduction processes, chemical distribution in (on or surrounding) the cell, timing control of connection between the cell and/or other cells, and/or connection strengths between the cell and other cells.

For instance with a neural cell, the cell has a certain threshold for stimulation, a time period where the cell cannot be stimulated again (absolute refractory period), a period where subsequent or continued stimulation will elicit a less intense secondary neural response (relative refractory period), a typical response pattern to stimulatory energy (such as a particular neural cell type could have its level of excitability increased (facilitated) or decreased (inhibited) relative to the stimulation frequency that might be applied to it), a particular orientation, and a particular position in the targeted tissue. The synchronization element can be used to synchronize the energy intensities such that the neural threshold for stimulation is reached from the combined energies (within a set period of time), an to apply the synchronized energies with a coordinated waveforms (of particular shapes) that are optimized to the targeted cell in a particular intensity or frequency, to apply the synchronized energies for the appropriate duration and/or reapplying the synchronized energies such that desired type of stimulation is elicited such as a single action potential or multiple action potentials, to apply the synchronized energies such that combined energy fields are directed along axis of the neural target in the appropriate direction (such as coordinating mechanical and electrical fields such that they are synchronously directed along the axon axis), and/or where the energies are synchronized to be maximum at a particular position relative to the neuron (such as the coordinate energies are synchronized to be maximum at a neural hillock).

For example, the synchronization element can be used to coordinate electrical and mechanical pulses that are synchronized to be turned on and become maximum in intensity at the same time to reach the neural threshold to initiate a neural spike (or synchronized such that the displacement current that is generated by their combined effects is maximum such as to reach the neural threshold to initiate a neural spike), which are then both synchronized to be turned off together within the period of the absolute refractory period of a neural cell, but reapplied again after the period of a the relative refractory period at a frequency that is known to elicit a facilitatory response from the neural cell. These fields could also in turn be synchronized to the changing ionic distributions that surround the cell (such as would be affected during the generation of an action potential) or the fields could be synchronized relative to the release of certain neurotransmitters from the cell or synchronized real time based on imaging modalities, physiological monitoring methods/devices, diagnostic methods/devices, and feedback methods/devices (such as those described in co-owned and co-pending U.S. patent application Ser. No. 13/162,047, the content of which is incorporated by reference herein in its entirety).

As another example the synchronization element, 33, can be used coordinate the fields such that the electrical and mechanical fields are out of phase with each other, such that the electrical field is at a maximum in intensity yet the mechanical field is maximum at the rate its intensity is changing in time, and such that the tissue impedance is maximally changing with time, and thus a maximum displacement current was generated in the location of a neural cell in order to stimulate the cell. The phase (and/or any relative property) between two energy waveforms can be altered due to the response dynamics of the targeted cell, tissue, network, organ, and/or organism.

The synchronization element, 33, could also be to affect or be synchronized with the function, distribution, and/or structure of elements surrounding the cell, such as for example: proteins, enzymes, macromolecules, ions, fluids, fluid concentrations, particles, genetic material, chemicals, transmitters, hormones, neural transmitters, and/or inflammatory elements.

As another example, the synchronization of energies can be applied to affect or be synchronized with tissue functions and/or processes, such as a tissue's: level of excitability (such as for example neural tissue the likelihood that a tissue is going to spontaneously generate action potentials, or the average stimulation threshold of the tissue (as could be determined from a calculation based on its cellular elements or based on the tissue as a whole)), blood flow in and out of the tissue, temperature of tissue, physiological processes intensity or timing, metabolic rates, glucose absorption rates, fluid concentrations, ionic distributions in tissue, and/or chemical concentrations in the tissue. As another example, the synchronization of energies can be applied to affect or be synchronized to a networks function or processes, such as for example a network's: level of excitability (of individual nodes or the entire network, such as for example affecting the excitability of the thalamus when using synchronized combined energies for adjusting the motor systems efficiency at learning new motor tasks, or in affecting the entire motor system by affecting every element of the motor system), connection strengths between individual nodes of the network or the entire network, and/or the timing of processes and/or communication between the individual nodes of the network. As another example, the synchronization of energies can be applied to affect or be synchronized with organ functions and/or processes, such as for example: timing of physiological processes (such as adjusting the rate at which an organ can process sensory information), intensity of physiological processes (such as adjusting the overall level of particular EEG band rhythms in the brain), release of chemicals from the organ (such as the release of neuroendocrine elements from the brain), communication to or from organ to other organs (such as adjusting the signal sent from the brain to the heart which controls its pace), and/or organ control of systemic processes (such as for the brain controlling inflammatory processes in the system and connected systems in the body). As another example, the synchronization of energies can be applied to affect or be synchronized to an organism's function or systemic activity (such as for example affecting a persons level of addiction, emotion, pain, alertness, and/or ability to learn).

The synchronization element, 33, can be integrated with controllers (such as for example to control the individual energies dynamic behavior, phase, direction, or intensity) of the individual sources (such as when one integrates separate energy sources to stimulate tissue, such as with an ultrasound device and an electrical stimulation device) or as a single element where a single source transducer provides both energies from a single element. Similarly, the synchronization element, 33, can be used to coordinate just one energy field at the time, such as for the above examples either the electrical field or mechanical field can be pulsed at the desired patterns while the other field is constantly provided.

Methods of the invention can be implemented during stimulation, after stimulation, or before stimulation (such as where synchronization planning can take place via simulation, such as with methods described in co-owned and co-pending U.S. patent application Ser. No. 13/216,282, the content of which is incorporated by reference herein in its entirety).

A chemical agent, optical, or thermal field can also be applied to the tissues to alter the permittivity of the tissue relative to an applied electric field. These methods can also be used in combination to alter the tissue permittivity relative to an applied electric field via invasive or noninvasive methods.

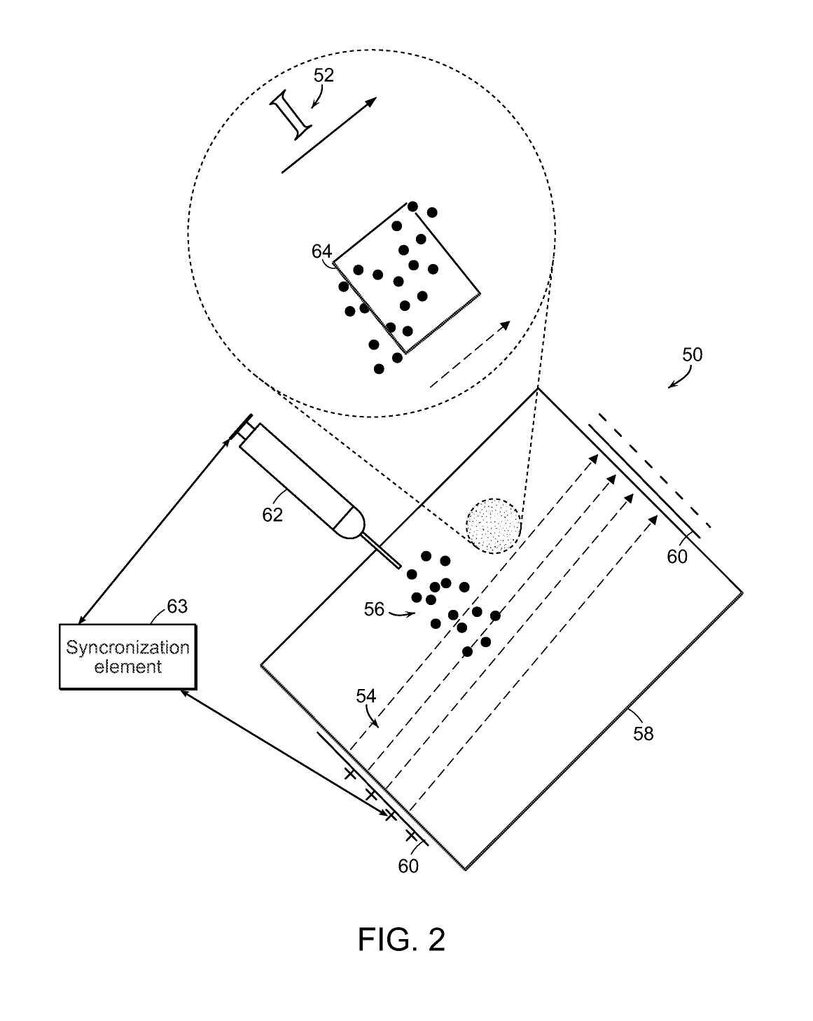

For example, FIG. 2 shows a set up 50 for generating an altered current with a newly generated displacement current 52 through the combined effects of an electric field 54 and a chemical agent 56. A tissue or composite of tissues 58 is placed within an electric source 60 which generates an electric field 54 and combined with chemical source 62 which releases a chemical agent 56 that can be focused on the tissue 58. A synchronization element, 63, is implemented to coordinate the application of the electrical and chemical energies (e.g., the chemical agent). In the area that the chemical agent 56 is released in the tissue 64, the electric field 54 transects the sub region of tissue 64, and the chemical agent 56 reacts with the sub region of tissue 64 to alter the tissue's relative permittivity relative to the applied electric field 54. This generates a displacement current 52 in addition to the current that would be present due to the source electric field 54. The chemical agent 56 may be any agent which can react with the tissue or cellular components of the tissue 64 to alter its permittivity relative to the electric field 54. This may be by a thermoreactive process to raise or lower the tissue 64 temperature or through a chemical reaction which alters the distribution of ions in the cellular and extra-cellular media, for instance, along ionic double layers at cell walls in the tissue 64. Similarly, the conformation of proteins and other charged components within the tissue 64 can be altered such that the permittivity of the tissue is altered relative to the low frequency electric field 54. The agent can also be any agent that adapts the permanent dipole moments of any molecules or compounds in the tissue 64, temporarily or permanently relative to the low frequency electric field 54. The chemical reaction driven by the chemical agent 56 must work rapidly enough such that the permittivity of the tissue is quickly altered in the presence of the electric field 54 in order to generate the displacement current 52. The reaction may also be such as to fluctuate the permittivity, such that as the permittivity continues to change displacement currents continue to be generated. In addition to the main permittivity change that occurs in the tissues, a conductivity change can also occur in the tissue, which secondarily alters the ohmic component of the currents. A biological agent may be used in place of, or in addition to, the chemical agent 56. This embodiment may have particular application for focused drug delivery where an additional chemical or biological agent is included to assist in therapy of the tissue, or where the altered current can drive an additional electrochemical reaction for therapy. For example, this can be used in areas such as focused gene therapy or focused chemotherapy.

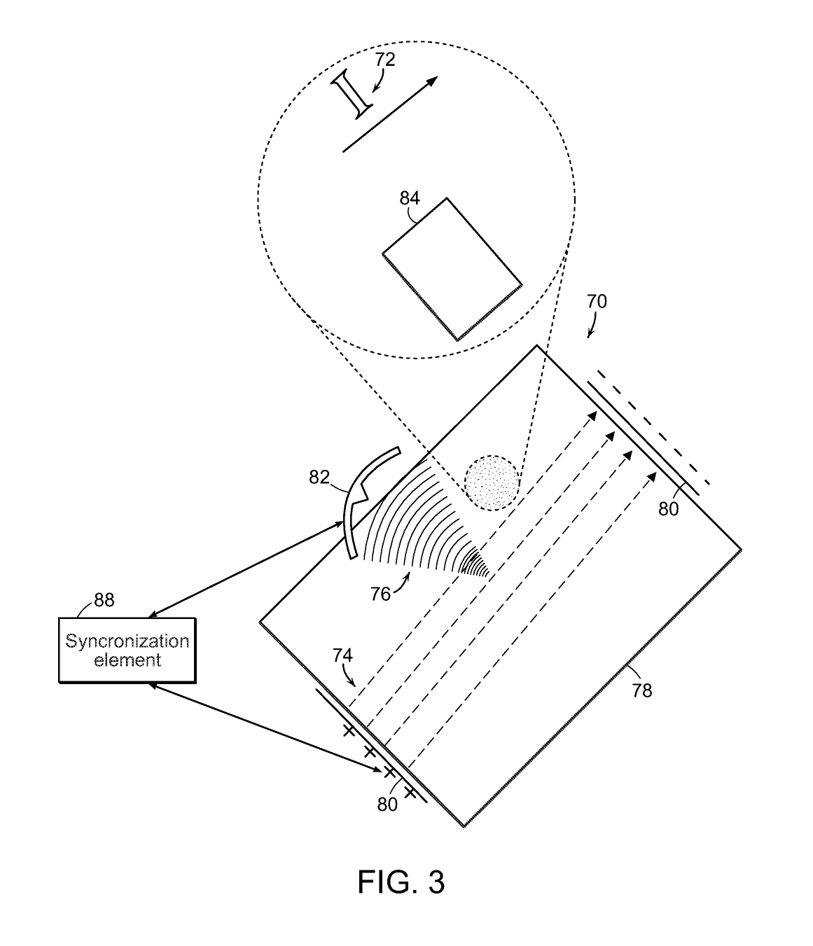

Another example is shown in FIG. 3, which illustrates a set up 70 for applying a method for generating an altered current with a newly generated displacement current 72 through the combined effects of a low frequency electric field 74 and an electromagnetic radiation field 76. A tissue or composite of tissues 78 is placed within a low frequency electric field 74 which is generated by an electric source 80 and combined with radiation source 82 which generates a radiation field 76 that can be focused on the tissue 78. The synchronization of the energies is accomplished by the synchronization element, 88, which is coordinating the radiation source, 82, which generates a radiation field, 76, and an electric source, 80, which is generating a low frequency electric field, 74. In the area that the radiation field 76 is focused in the tissue 78, the electric field 74 transects the sub component of tissue 84, where the radiation field 76 interacts with the sub component of tissue 84 to alter the tissue's relative permittivity relative to the applied electric field 74, and as such generates a displacement current 72 in addition to the current that would be present due to the source electric field 74 or the radiation source field 76 alone. The electromagnetic radiation field 76 can, for example, interact with the tissue 84 by altering its temperature through ohmic processes, alter the distribution of ions in the cellular and extra-cellular media for instance along ionic double layers along cell walls through the electric forces acting on the ions, or alter the conformation of proteins and other charged components within the tissue through the electric forces such that the permittivity of the tissue is altered relative to the low frequency electric field 74. Furthermore, the electromagnetic field 76, can interact with the tissue 84 by moving components of the tissue via electrorestrictive forces, as would be seen in anisotropic tissues, to alter the continuum permittivity of the tissue relative to the low frequency electric field 74. In addition to the main permittivity change that occurs in the tissues, a conductivity change can also occur in the tissue, which secondarily alters the ohmic component of the currents.

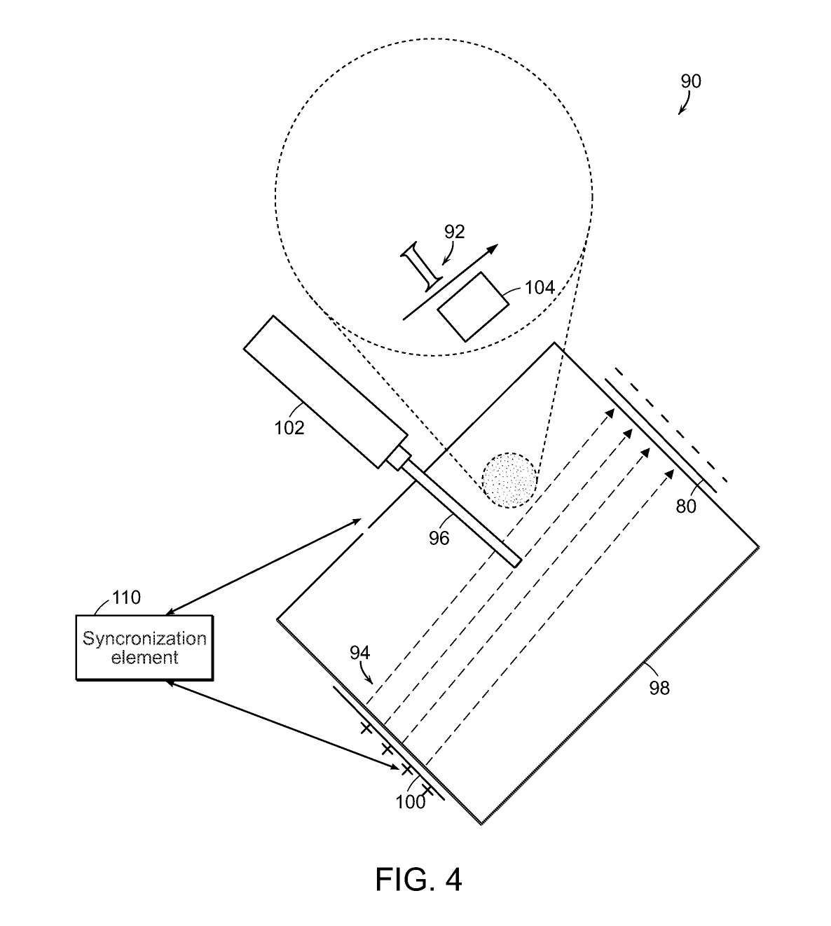

FIG. 4 shows a set up, 90, for applying a method for generating an altered current with a newly generated displacement current, 92, through the combined effects of an electric field, 94, and an optical beam, 96. A tissue or composite of tissues, 98, is placed within electric field, 94, generated by an electric source, 100, and combined with optical source, 102, which generates optical beam, 96, that can be focused on the tissue, 98. A synchronization element, 110, is used to synchronize the electrical and optical energies. In the area that the optical beam 96 is focused on the tissue, the electric field 94 transects the sub component of tissue 104, where the optical beam 96 reacts with the tissue to alter the tissue's relative permittivity relative to the applied electric field 94, and as such generates a displacement current 92 in addition to the current that would be present due to the source electric field 94. The optical beam 96 can, for example, interact with the tissue by altering its temperature through photothermal effects and/or particle excitation, alter the distribution of ions in the cellular and extra-cellular media for instance along ionic double layers along cell walls by exciting the movement of ions optically, ionizing the tissue via laser tissue-interactions, or alter the conformation of proteins and other charged components within the tissue such that the permittivity of the tissue is altered relative to the low frequency electric field 94. In addition to the main permittivity change that occurs in the tissues, a conductivity change can also occur in the tissue, which secondarily alters the ohmic component of the currents.

In another embodiment, a thermal source to alter the permittivity of the tissue may be used. In such embodiments, a thermal source such as a heating probe, a cooling probe, or a hybrid probe may be placed external or internal to the tissue to be stimulated. A thermal source may alter the permittivity of the tissue through the direct permittivity dependence of tissue temperature, mechanical expansion of tissues in response to temperature changes, or by mechanical forces that arise due to altered particle and ionic agitation in response to the temperature alteration such that permittivity of the tissue is altered relative to an applied electric field. In addition to the main permittivity change that occurs in the tissues, a conductivity change can also occur in the tissue, which secondarily alters the ohmic component of the currents. A synchronization element is used in this embodiment to synchronize the thermal and electrical energies. This embodiment may be useful for stimulation in the presence of an acute injury to the tissue where the thermal source can be used to additionally assist in the treatment of the tissue injury, for example with a traumatic brain injury or an infarct in any organ such as the heart. The tissue can be cooled or heated at the same time stimulation is provided to reduce the impact of an injury.

In a further embodiment, the method according to the present disclosure is applied in the area of muscular stimulation, where amplified, focused, direction altered, and/or attenuated currents and/or synchronized combined energies can be used to alter muscular activity via direct stimulation, depolarizing muscular cells, hyperpolarizing muscular cells, modifying membrane potentials, and/or increasing or decreasing the excitability of the muscle cells. This alteration of excitability or firing patterns can last past the duration of stimulation and thus be used as a basis to provide lasting treatment. Additionally, the stimulation can be provided in multiple, but separate sessions to have a summed, or carry-over effect, on the excitability of cells and tissue. Additionally, the stimulation can be provided to prime the tissue by adjusting the muscle cell excitability to make it more or less susceptible to alternate follow up forms of stimulation. The stimulation can be used after another form of stimulation was used to prime the tissue. Furthermore, the stimulation can be applied for a chronic period of time. This embodiment may be useful for altering or assisting cardiac pacing or function, assisted breathing, muscle stimulation for rehabilitation, muscle stimulation in the presence of nerve or spinal cord injury to prevent atrophy or assist in movement, or as substitution for physical exercise.

In yet another embodiment, the method according to the present disclosure can be applied the area of physical therapy, where amplified, focused, direction altered, and/or attenuated currents and/or synchronized combined energies can be used to stimulate blood flow, increase or alter neuromuscular response, limit inflammation, speed the break down of scar tissue, and speed rehabilitation by applying the focus of the current generation to the effected region in need of physical therapy. It is envisioned that the method according to the present disclosure may have a wide variety in the area of physical therapy including the treatment or rehabilitation of traumatic injuries, sports injuries, surgical rehabilitation, occupational therapy, and assisted rehabilitation following neural or muscular injury. For instance, following an injury to a joint or muscle, there is often increased inflammation and scar tissue in the region and decreased neural and muscular response. Typically, ultrasound is provided to the affected region to increase blood flow to the region and increase the metabolic re-absorption of the scar tissue while electrical stimulation is provided separately to the nerves and muscles; however, by providing them together, a person can receive the benefit of each individual effect, but additionally amplified stimulatory and metabolic effects through the altered currents. The other methods for generating altered currents discussed within can also be used to assist in physical therapy via the displacement currents that are generated.

Furthermore, the method according to the present disclosure may be applied to the area of cellular metabolism, where currents and/or synchronized energies can be used to interact with energy receptive cells or membranes to alter the tissue or cellular dynamics.

Furthermore, the method according to the present disclosure may be applied to the area of gene therapy. Amplified, focused, direction altered, and/or attenuated currents and/or synchronized combined energies can be used to interact with energy receptive cells or receptors within the cell to influence protein transcription processes and alter the genetic content of the cells. The altered current densities and/or combined energies in the tissue can interact with the tissue to stimulate this altered gene regulation. Additionally, the displacement currents and/or combined energies generated by the method can further be used to assist in drug delivery and/or gene therapy through the altered current influence on the delivery of agents.

Furthermore, the method according to the present disclosure may be applied to the area of tissue growth. Synchronized combined energies can be used to interact with cells or receptors within the cell to influence cell growth (and/or tissue(s)) and/or alter cellular (and/or tissue(s)) processes and/or structures. Stimulation can be used to increase and or slow cell and/or tissue growth and/or affect tumors, such as through ablation methods whereby energies can be used to ablate tissues or with methods whereby combined energies can create tumor treating fields. The synchronization element can be applied to coordinate the combined energies. For instance, with electromechanical stimulation, altered electromagnetic fields generated via electromechanical coupling can affect the tissues, while in certain embodiments the mechanical fields can also have a further therapeutic effect where the relative timing between the fields is synchronized by the synchronization elements.

For the devices and methods disclosed herein, synchronization can be used to generate multiple effects that can be tuned in any combination (based or not based on the current generation in the tissue); such as for example with multiple cellular effects of stimulation, one for example can synchronize two energy fields such that the effects of one energy on a cellular function is enacted independent of the second energy (such as an electrical field applied to open voltage dependent channels on a cellular membrane), while another energy acts independently on a second cellular function (such as a mechanical field altering the electrical impedance of the axon membrane), and the effects of the combined energy on a third cellular function (such as the electromechanical pulse generates an increase in ion flow which initiates an action potential in the cell). This synchronization of the fields can thus be tuned relative to function of the targeted cell(s), tissue(s), network(s), organ(s), organism(s) (where the effects can be independent, symbiotic, and/or have a cancelling effect on each other).

Furthermore, the combined fields can be provided, where individually the fields have one particular effect, but together they enact effects different (or greater) than either of the individual energies' effects. The fields can be applied where one of the fields might inhibit cellular function and the other field facilitates cellular function, but their combined effects have one or the other effects (inhibitory or facilitatory) which can be greater than any of the individual effects (such as those outlined in (U.S. patent application Ser. No. 13/216,313)).

Additionally, the examples herein were provided with two energy sources, but the synchronization elements can be applied with any number or combination of stimulation energies.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

The invention may be embodied in other specific forms without departing from the spirit or essential characteristics thereof. The foregoing embodiments are therefore to be considered in all respects illustrative rather than limiting on the invention described herein.

What is claimed is:

1. A system for stimulating tissue, the system comprising:
a first energy source;
a second energy source; and
a synchronization element that synchronizes the first and second energy sources, wherein the combined effect stimulates the tissue.

2. The system according to claim 1, wherein the first energy source is an electric source that produces an electric field.

3. The system according to claim 2, wherein the second energy source is a source that produces a mechanical field.

4. The system according to claim 3, wherein the second energy source is an ultrasound device.

5. The system according to claim 2, wherein the electric field is pulsed.

6. The system according to claim 2, wherein the electric field is time varying.

7. The system according to claim 2, wherein the electric field is pulsed a plurality of time, and each pulse may be for a different length of time.

8. The system according to claim 2, wherein the electric field is time invariant.

9. The system according to claim 3, wherein the mechanical field is pulsed.

10. The system according to claim 3, wherein the mechanical field is time varying.

11. The system according to claim 3, wherein the mechanical field is pulsed a plurality of time, and each pulse may be for a different length of time.

12. The system according to claim 2, wherein the electric field is focused.

13. The system according to claim 3, wherein the mechanical field is focused.

14. The system according to claim 3, wherein both the electric field and the mechanical field are focused.

15. The system according to claim 1, wherein the first and second energy sources are applied to a structure or multiple structures within the brain or the nervous system selected from the group consisting of: dorsal lateral prefrontal cortex, any component of the basal ganglia, nucleus accumbens, gastric nuclei, brainstem, thalamus, inferior colliculus, superior colliculus, periaqueductal gray, primary motor cortex, supplementary motor cortex, occipital lobe, Brodmann areas 1-48, primary sensory cortex, primary visual cortex, primary auditory cortex, amygdala, hippocampus, cochlea, cranial nerves, cerebellum, frontal lobe, occipital lobe, temporal lobe, parietal lobe, sub-cortical structures, and spinal cord.

16. The system according to claim 1, wherein the tissue is neural tissue.

17. The system according to claim 16, wherein the affect of the stimulation alters neural function past the duration of stimulation.

18. The system according to claim 1, wherein the synchronization element synchronizes the first and second energy in relative: energy magnitude, energy position, energy dynamic behavior (i.e., behavior as a function of time), energy static behavior, energy behavior in the frequency domain, energy phase, orientation/direction of energy fields (i.e., vector behavior), duration of energy application (in single or multiple sessions), and/or composition of energy.

19. The system according to claim 1, wherein the synchronization element synchronizes the energies relative timing based on cell function.

20. A method for stimulating tissue, the method comprising:
providing a first type of energy to a region of tissue;
providing a second type of energy to the region of tissue; and
synchronizing the first and second energy types, wherein the combined effect stimulates the tissue.

21. The method according to claim 20, wherein the first type of energy is a mechanical field.

22. The method according to claim 21, wherein the mechanical field is generated by an ultrasound device.

23. The method according to claim 21, wherein the mechanical field is pulsed.

24. The method according to claim 21, wherein the mechanical field is time varying.

25. The method according to claim 21, wherein the mechanical field is pulsed a plurality of time, and each pulse may be for a different length of time.

26. The method according to claim 20, wherein the second type of energy is an electric field.

27. The method according to claim 26, wherein the electric field is pulsed.

28. The method according to claim 26, wherein the electric field is time varying.

29. The method according to claim 26, wherein the electric field is pulsed a plurality of time, and each pulse may be for a different length of time.

30. The method according to claim 26, wherein the electric field is time invariant.

31. The method according to claim 20, wherein the first and second types of energy are applied to a structure or multiple structures within the brain or the nervous system selected from the group consisting of: dorsal lateral prefrontal cortex, any component of the basal ganglia, nucleus accumbens, gastric nuclei, brainstem, thalamus, inferior colliculus, superior colliculus, periaqueductal gray, primary motor cortex, supplementary motor cortex, occipital lobe, Brodmann areas 1-48, primary sensory cortex, primary visual cortex, primary auditory cortex, amygdala, hippocampus, cochlea, cranial nerves, cerebellum, frontal lobe, occipital lobe, temporal lobe, parietal lobe, sub-cortical structures, and spinal cord.

32. The method according to claim 20, wherein the tissue is neural tissue.

33. The method according to claim 32, wherein the affect of the stimulation alters neural function past the duration of stimulation.
